(19) United States
(12) Patent Application Publication
(10) Pub. No.: US 2005/0203487 A1
(43) Pub. Date: Sep. 15, 2005

(54) Lubrication system for a powered surgical instrument

(75) Inventors: Bryan D. Simmons, Flower Mound, TX (US); John K. Henderson, Flower Mound, TX (US); Durrell Tidwell, Burleson, TX (US); Cameron McPherson, Fort Worth, TX (US)

Correspondence Address: HAYNES AND BOONE, LLP, 901 MAIN STREET, SUITE 3100, DALLAS, TX, 75202, US

(73) Assignee: Medtronic, Inc., Minneapolis, MN (US)

(21) Appl. No.: 10/699,299

(22) Filed: Oct. 31, 2003

(51) Int. Cl. A61B 17/00

(52) U.S. Cl. 606/1

(57) ABSTRACT

A lubrication system for a pneumatically powered instrument is disclosed. In one example, a lubrication system for use with a surgical instrument includes: a first fluid path comprising a first fluid inlet, and a first fluid outlet wherein the first fluid outlet does not contact a lubricant; and a second fluid path comprising a second fluid inlet extending into the lubricant, a filtering system, and a controlled path, and a pressurized fluid flows through the first fluid path, causing at least a portion of the lubricant to flow through the second fluid

### CROSS REFERENCE

This application is related to the following U.S. patent applications: U.S. Ser. Nos. 60/301,491 filed on Jun. 28, 2001; 60/352,609 filed on Jan. 28, 2002; 60/360,332 filed on Feb. 26, 2002; and 10/180,47 filed on Jun. 26, 2002, all of which are hereby incorporated by reference.

### FIELD OF THE INVENTION

The present invention generally relates to surgical instruments. More particularly, the present invention relates to powered surgical instruments and a lubrication system for use therewith.

### BACKGROUND

Doctors and other medical professionals often use powered surgical instruments for dissecting bones, tissues and other purposes. Frequently, it is important to lubricate the instruments for proper usages. For example, a pneumatically powered surgical instrument may include a pneumatic motor that is connected to a fluid supply source, and a lubrication system is typically placed inline between the fluid supply source and the pneumatic motor to provide lubrication to the surgical instrument.

Traditionally, the lubrication system must be manually calibrated and/or activated according to predetermined guidelines. For example, the lubrication system is typically set at a specific dripping rate for providing oil to the surgical instrument. Therefore, to supply a certain amount of lubrication to the instrument, it is important to maintain a proper dripping rate. However, since the manual operation is prone to mistakes and inaccuracy, the amount of supplied oil varies erratically, and too much or too little oil may be provided to the instrument.

Therefore, it is desired to provide an improved lubrication system that supplies consistent lubrication to a surgical instrument. Is further desired to provide an improved lubrication system that does not require frequent manual operations.

### SUMMARY

The present invention provides an improved lubrication system for a surgical instrument.

In one embodiment, a lubrication system for use with a surgical instrument comprises: a first fluid path comprising a first fluid inlet, and a first fluid outlet wherein the first fluid outlet does not contact a lubricant; and a second fluid path comprising a second fluid inlet extending into the lubricant, a filtering system, and a controlled path, wherein a pressurized fluid flows through the first fluid path, causing at least a portion of the lubricant to flow through the second fluid path.

In a second embodiment, A surgical system comprising a pneumatically powered surgical instrument with a motor, and a lubrication system for providing lubrication to the motor, wherein the lubrication system comprises: a first enclosure positioned inline with a pressurized fluid path, the first enclosure comprising a large-diameter channel and a small-diameter channel; a second enclosure including a lubricant; a first fluid path comprising a first fluid inlet coupled with the large-diameter channel, and a first fluid outlet; and a second fluid path comprising a second fluid inlet extending into the lubricant, a filtering system, a controlled path, and a second fluid outlet coupled with the small-diameter channel, wherein at least a portion of a pressurized fluid flows from the large-diameter channel and through- the first fluid path, causing at least a portion of the lubricant to flow through the second fluid path and into the small-diameter channel.

In a third embodiment, an inline oiler for use with a pneumatically powered surgical instrument comprises: a first inlet for receiving pressurized air; a first outlet for providing the pressurized air into an enclosure wherein the enclosure comprises a lubricant, wherein the first outlet does not contact the lubricant; a second inlet for receiving at least a portion of the lubricant wherein the second inlet extends into the lubricant; a filtering system for filtering the at least a portion of the lubricant; and a capillary tube for controlling the flow of the filtered lubricant.

It should be understood that the present summary and the following detailed description, while indicating embodiments of the invention, are intended for purposes of illustration only and are not intended to limit the scope of the invention beyond that described in the claims.

### BRIEF DESCRIPTION OF THE DRAWINGS

The present disclosure will become more fully understood from the detailed description and the accompanying drawings, wherein:

FIG. 1A illustrates a lubrication system for a pneumatically powered instrument according to one embodiment of the present disclosure.

FIG. 1B is a perspective view of the lubrication system of FIG. 1A.

FIG. 2A is a side view of a partial lubrication system for a pneumatically powered instrument according to one embodiment of the present disclosure.

FIG. 2B is a perspective view of a partial lubrication system for a pneumatically powered instrument according to one embodiment of the present disclosure.

FIG. 2C is a top view of a partial lubrication system for a pneumatically powered instrument according to one embodiment of the present disclosure.

FIG. 2D is a bottom view of a partial lubrication system for a pneumatically powered instrument according to one embodiment of the present disclosure.

FIG. 2E is a cross-sectional view taken along the line 2E-2E of FIG. 2D.

FIG. 2F is a cross-sectional view taken along the line 2F-2F of FIG. 2D.

FIG. 2G is a side view of a first conduit of a lubrication system for a pneumatically powered instrument according to one embodiment of the present disclosure.

FIG. 2H is a cross-sectional view taken along the line 2H-2H of FIG. 2G.

FIG. 3 is a cross-sectional view of a lubrication system for a pneumatically powered instrument according to one embodiment of the present disclosure.

FIG. 4A is an exploded view of an injector system of from the lubrication system of FIG. 3.

FIG. 4B is a perspective view of assembled components selected from FIG. 4A.

FIG. 4C is a perspective view of the assembled components of FIG. 4B rotated by 180 degrees.

### DETAILED DESCRIPTION

For the purposes of promoting an understanding of the principles of the invention, references will now be made to the embodiments, or examples, illustrated in the drawings and specific languages will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Further, it will be understood that the present disclosure is not limited to any particular surgical application but has utility for various applications in which it is desired, including but not limited to:

1. Arthroscopy—Orthopaedic

2. Endoscopic—Gastroenterology, Urology, Soft Tissue

3. Neurosurgery—Cranial, Spine, and Otology

4. Small Bone—Orthopaedic, Oral-Maxiofacial, Ortho-Spine, and Otology

5. Cardio Thoracic—Small Bone Sub-Segment

6. Large Bone—Total Joint and Trauma

7. Dental and other applications

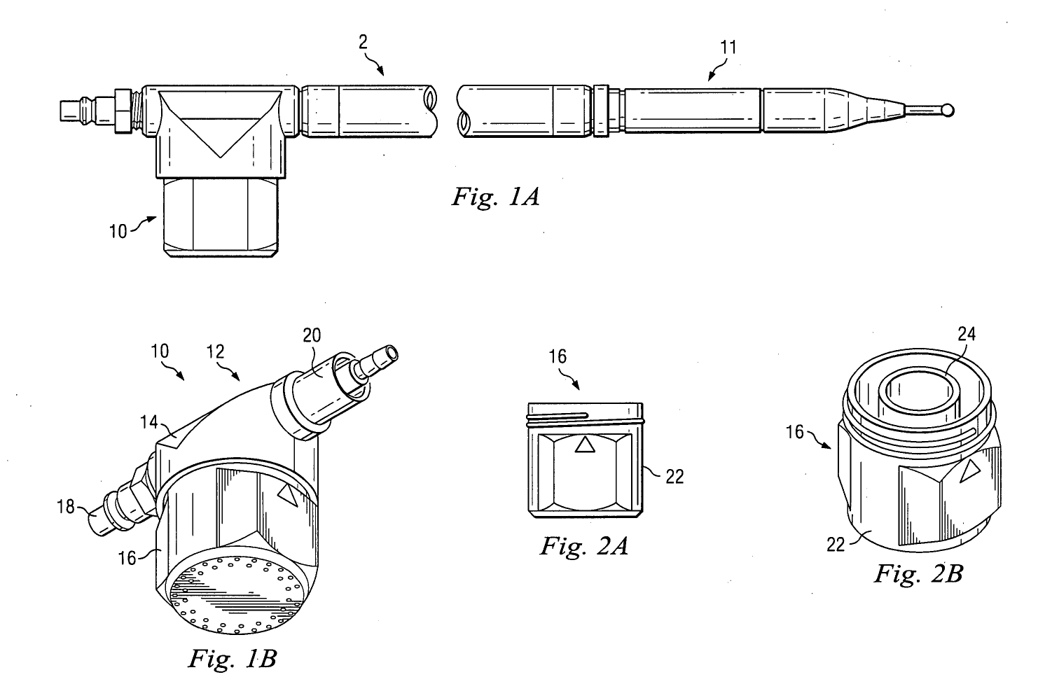

Referring now to FIG. 1A, shown therein is a lubrication system 10 for a pneumatically powered surgical instrument according to one embodiment of the present disclosure. In this illustration, the lubrication system 10 may be used with a pneumatically powered surgical instrument 11. An exemplary surgical instrument 11 is shown in a commonly assigned U.S. Pat. No. 5,505,737, which is hereby incorporated by reference as if fully set forth herein. It is contemplated that the teachings of the present disclosure also apply to other powered instruments.

As lubrication systems have been fully described in the U.S. patent application Ser. No. 10/180,470 filed on Jun. 26, 2002 (which is hereby incorporated by reference), many components of the lubrication system 10 will only be briefly described herein.

Referring now to FIG. 1B, in one embodiment, the lubrication system 10 may include a body or housing 12, a first conduit 18 for delivering a source of pressurized air to the pneumatically powered instrument 11, and a second conduit 20 for returning exhaust gases from the pneumatically powered instrument 11 to the housing 12 of the lubrication system 10. Although the first and second conduits 18, 20 are coaxial as illustrated in FIG. 1B, it is understood that in other embodiments, those conduits may be separate (not coaxial). It is also contemplated that the lubrication system 10 may comprise a single conduit or more than two conduits.

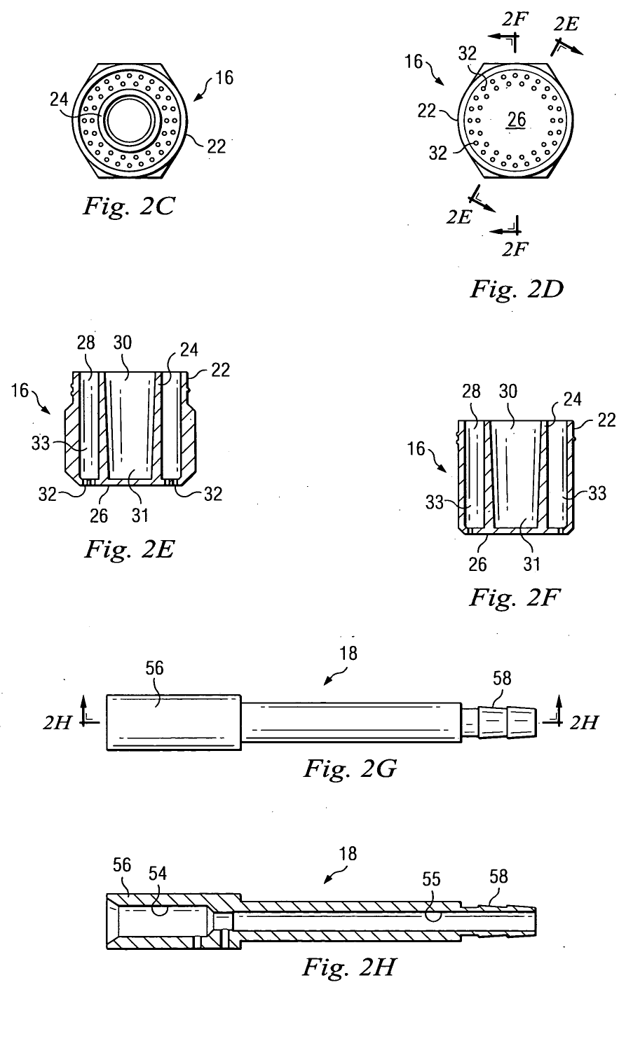

The lower portion of the lubrication system 10 will now be further described. Referring now to FIGS. 2A-2F, in one embodiment, a lower portion 16 of the housing 12 may comprise a generally cylindrical shape. In one application, the lower portion 16 of the housing 12 may comprise plastics. Alternatively, the lower portion 16 may comprise any other suitable material, such as stainless steel, titanium, shape memory alloys, polymers, carbon fiber, and porous material.

In this example, the lower portion 16 may include an outer cylindrical wall 22, a concentrically arranged inner cylindrical wall 24, and a bottom wall 26. A first generally cylindrical cavity 28 may be defined between the inner and outer cylindrical walls 22 and 24. A second generally cylindrical cavity 30 may be defined by the inner cylindrical wall 24.

In furtherance of the example, as shown in FIG. 2F, an oil saturated cellulose fiber material 31 may be disposed in the second cavity 30. Also, a dry cellulose fiber material 33 may be disposed within the first cavity 28. It will be understood that the cellulose fiber materials 31, 33 are merely examples of absorbent materials, and other materials, such as foam, wool felt, porous plastics, porous metals and/or other type materials, are also contemplated. As described below, oil may be drawn from the second cavity 30 and into a flow stream. Exhaust gases, which may include spent oil, may be returned to the first cavity 28. The dry cellulose fiber material 33 may filter the oil from the exhaust gases. The filtered exhaust gases are permitted to pass through a plurality of exhaust apertures 32 in the bottom wall 26 of the lower portion 16.

The upper portion of the lubrication system 10 will now be further described. Referring now to FIGS. 2G and 2H, shown therein is the first conduit 18 of the lubrication system 10 according to one embodiment of the present disclosure. In this embodiment, the first conduit 18 may be a hollow tubular member, which may comprise plastics or other suitable materials. The first conduit 18 may define a central channel, and a first end 56 of the first conduit 18 may be coupled to an air source (not shown). A second end 58 of the first conduit 18 is reduced in diameter and may be coupled to the pneumatically powered instrument 11 through a hose (not shown).

As particularly shown in FIG. 1B, in one embodiment, the second conduit 20 may be a hollow tubular member. The second conduit 20 may be received within the upper portion 14 of the housing 12 and cooperate with the upper portion 14 to define a fluid path for returning exhaust gases from the pneumatically powered instrument 11 to the outer cylindrical cavity 28 of the lower portion 16. The second conduit 20 may concentrically surround the second end 58 of the first conduit 18.

In an exemplary operation, pressurized air may be introduced into the first end 56 of the first conduit 18. In one particular application, the air may be introduced at a pressure of approximately 120 psi. Further, an on/off control mechanism, such as a foot pedal, may be disposed between a compressed air source and the instrument 11, such as at the first end 56 of the first conduit 18. The pressurized air may pass through the channel 54 defined by the first conduit 18, and draw oil from the wet fiber cellulose material 31 in the chamber 30 into the air stream. As a result, the oil is delivered with the air stream into the motor of the pneumatically powered instrument for lubrication.

Referring back to FIG. 1B and FIGS. 2E-2F, in furtherance of the exemplary operation, exhaust gases carrying spent oil from the motor of the pneumatically powered instrument may be returned through the second conduit 20. These exhaust gases may be introduced into the outer cylindrical chamber 28 containing the dry fiber cellulose material 33 through a pathway (not shown) defined in the upper portion 14 of the housing 12. The dry fiber cellulose material 33 in the cavity 28 may filter the spent oil from the exhaust gases and allow the exhaust gases to pass through the plurality of apertures 32 in the bottom wall 26.

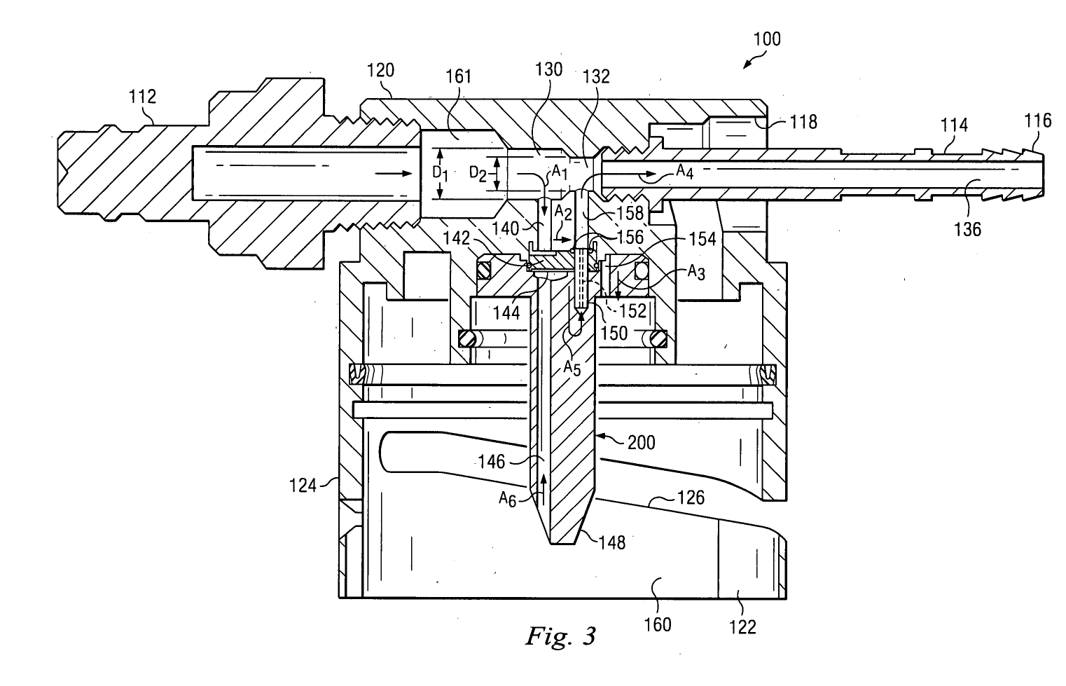

Referring now to FIG. 3, shown therein is a lubrication system 100 according to one embodiment of the present disclosure. The lubrication system 100 may include a body 120, which may comprise an inlet tube 112 and an outlet tube 114. The inlet tube 112 may be connected to a source of pressurized fluid, which may be pressurized air. As shown in FIG. 1B, a coaxial hose may be connected to the lubrication system 100—a higher pressure hose may be coupled with flanges 116, and a lower pressure exhaust hose may be coupled with an aperture 118. The body 120 may comprise a shell 124, which may be substantially cylindrical or in other suitable shapes. The shell 124 may comprise an opening at one end to receive a lubrication fluid reservoir and exhaust filter unit, and one or more locking slots 126 adapted to receive projections on the exterior of the lubrication fluid reservoir and exhaust filter unit and to retain it within the body 120. Internal grooves 122 may extend from the opening to the locking slots 126, so that the projections may be advanced into the interior of the body 120. The locking slots 126 may comprise a helical path, which may help to advance the lubrication and filter unit into the body 120. Further, the locking slots 126 may include substantially flat portions at their termination, so that the lubrication and filter unit may be locked into position in the body 120. The body 120 may comprise aluminum or other suitable materials, such as stainless steel, titanium, shape memory alloys, polymers, carbon fiber, and porous material.

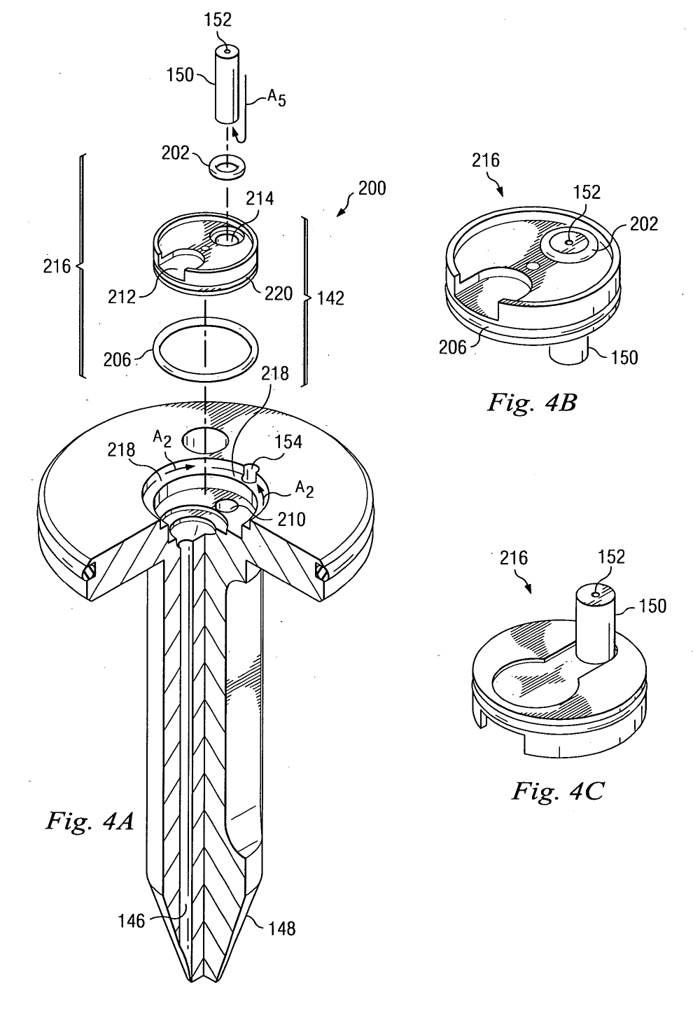

The lubrication system 100 may include fluid passageways adapted for providing pressurized fluid and lubrication to the surgical instrument. In one example, a first channel 130 having a diameter D1 may be in fluid communication with the pressurized fluid from the inlet tube 112. A second channel 132 with a reduced diameter D1 may provide a conduit between the first channel 130 and an outlet channel 136 within the outlet tube 114. Within the wall defining the first channel 130, an aperture may communicate with a first fluid inlet 140. Similarly, within the wall defining the second channel 132, an aperture may communicate with a second fluid outlet 158. An injector system 200 may be joined to the body 120 and substantially centered within the cylindrical shell 124. The injector system 200, which will be further described below in connections with FIGS. 4A-4C, may comprise a distribution system 142, a first fluid outlet 154, a second fluid inlet 146, a filtering system 144, and a tube 150 that includes a controlled path 152. The distribution system 142 may be in communication with the first fluid outlet 154, while the second fluid inlet 146 may be in communication with the filtering system 144, which in turn may be in communication with the tube 150. The filtering system 144 may be used to prevent large particles from entering into and clogging the controlled path 152. It may be a screen mesh or any other suitable filtering device, and each opening of the filtering system 144 may be smaller than the opening of the controlled path 152.

The lubricant 160 may simply reside in a chamber (not show). Alternatively, any absorbent material residing in the chamber, such as foam, wool felt, porous materials, or cellulose fiber materials, may be used to saturate the lubricant 160. In this example, the second fluid inlet 146 may be at least partially submerged in and compress the absorbent material (or the lubricant 160). However, it is also contemplated that the second fluid inlet 146 may not be submerged in the absorbent material. In addition, the first fluid outlet 154 may not directly contact the lubricant 160. However, it is also contemplated that the first fluid outlet 154 may contact the lubricant 160. The lubrication system 100 may be pre-filled with the lubricant 160. The amount of the lubricant 160 may be sufficient to ensure lubrication of the motor of the pneumatically powered instrument throughout a surgical procedure. Accordingly, when the lubrication system 100 functions properly, the risk of lacking the lubricant 160 during a surgical procedure may be effectively eliminated. After the surgical procedure is completed, the lubrication system 100 may be disconnected from the surgical instrument, and reused or discarded.

Referring also to FIGS. 4A and 4C, the present embodiment may incorporate a flow control mechanism that is accomplished at least in part by the controlled path 152. It will be understood that it is known in the art that partially based on the diameter and the length of the controlled path 152, and the viscosity of the lubricant 160, the volume of the lubricant 160 flowing through the controlled path 152 and into the surgical instrument may be calculated and determined. Therefore, to supply lubrication to a variety of surgical instruments, a great range of the diameter and length of the controlled path 152 is anticipated. In one example, the controlled path 152 may be a capillary tube. In a specific example, the diameter of the controlled path 152 may be 1/5000 inch, and the length of the controlled path 152 may be 1/10 inch. However, it is contemplated that other diameter and/or length figures for the controlled path 152 are also contemplated.

The injector system 200 will now be further described. Referring now to FIGS. 4A, shown therein is the injector system 200 according to one embodiment of the present disclosure. In this embodiment, the injector system 200 may comprise an upper portion 216 and a lower portion 148. The upper portion 216 may comprise the tube 150, an 0 ring 202, a distributor 220 that comprises an opening 214 adapted for receiving the tube 150, and an o ring 206. The lower portion 148 may comprise an opening 210 adapted for receiving the upper portion 216, the second fluid inlet 146, and the first fluid outlet 154. The assembled upper portion 216 is shown in FIGS. 4B-4C. The tube 150 may be loosely coupled with the opening 210, so that the lubricant 160 may travel between them as indicated by an arrow A5.

In operation, the lubrication system 100 may include air and lubricant paths. In one embodiment, the air path may comprise the first channel 130, the first fluid inlet 140, the distribution system 142, and the first fluid outlet 154. The lubricant path may comprise the second fluid inlet 146, the filtering system 144, the controlled path 152, the second fluid outlet 158, and the second channel 132. As indicated previously, the first channel 130 has the diameter D1, which may be larger than the diameter D2 of the second channel 132.

In furtherance of the example, high pressurized air, which may be at a pressure of approximately 120 psi, may be generated from a source. An on/off control mechanism, such as a foot pedal, may be disposed between a compressed air source and the surgical instrument. The pressurized air may flow through a chamber 161 and into the first channel 130, and then encounter restriction at the smaller second channel 132. As indicated by arrows A1-A3, a portion of the high pressurized air may enter the first fluid inlet 140, travel through the distribution system 142, the operation of which will be further described below in connections with FIGS. 4A-4C, and exit through the first fluid outlet 154. As the high pressurized air exits from the first fluid outlet 154, it may cause the lubricant 160, which may be oil or any other suitable lubrication fluid, to enter into the second fluid inlet 146 and travel in the direction indicated by an arrow A6. There, the lubricant 160 may encounter the filtering system 144, the operation of which will be further described below. The filtering system 144 may stop undesirable large particles mixed with the lubricant 160, and allow the filtered lubricant 160 to travel downward and along the exterior of a tube 150, and then upward into the controlled path 152 as indicated by an arrow A5. Once inside the controlled path 152, the lubricant 160 may flow into and through the second fluid outlet 158, and exit into the second channel 132. As indicated by an arrow A4, the lubricant 160 may then travel through the outlet channel 136 along with the high pressurized air from the channel 132, so that the lubricant 160 may be used by a surgical instrument. It will be understood that in this illustration, the first fluid inlet 140 may have a lower altitude than that of the controlled path 152, and that the first fluid outlet 154 may have a higher altitude than that of the second fluid inlet 146. However, other arrangements of relative altitudes are also contemplated.

The operation of the distribution system 142 will not be further described. Referring now to FIG. 4A, in one embodiment, the distribution system 142, which may comprise the distributor 200, the O ring 206, and a substantially circular path 218, may direct the pressurized air in the following manner: the pressurized air may travel to the indented component 212, exit from the distributor 220, then continue along the substantially circular path 218 as indicated by the arrows A2, and finally exit into the first fluid outlet 154. It is contemplated that the distribution system 142 may employ other means to direct pressurized fluid from the first fluid inlet 140 to the first fluid outlet 154. In one example, the distribution system 142 may be a channel between the first fluid inlet 140 and the first fluid outlet 154. In another example, the first fluid inlet 140 may simply be in fluid communication with the first fluid outlet 154.

The operation of the filtering system 144 will now be further described. As indicated by the arrow A5 of FIG. 4A, the lubricant 160 may travel from the filtering system 144 of FIG. 3, continue along the space between the tube 150 and the opening 210, and then travel into the controlled path 152.

Generally, when a lubrication system fails to provide a proper amount of lubrication to the surgical instrument, it is preferable to supply zero or very limited amount of lubrication. Therefore, the lubrication system 100 may employ a number of features to cope with a system failure, and to prevent the surgical instrument from being overwhelmed with an excessive amount of the lubricant. Referring back to FIG. 3, in one example, when the controlled path 152 becomes clogged and is no longer able to effectively control the flowing amount of the lubricant, it simply decreases the volume of the lubricant that flows into the surgical instrument. In another example, a seal 156, which may be an O ring, is utilized, so that high pressurized air from the distribution system 142 will not be improperly diverted into the second fluid outlet 158. However, when the seal 156 fails, the high pressurized air may simply enter into the second fluid outlet 158. As a result, a decreased amount of the lubricant will be provided to the surgical instrument.

Although only a few exemplary embodiments of this invention have been described in detail above, those skilled in the art will readily appreciate that many modifications are possible in the exemplary embodiments without materially departing from the novel teachings and advantages of this invention. Also, features illustrated and discussed above with respect to some embodiments can be combined with features illustrated and discussed above with respect to other embodiments. Accordingly, all such modifications are intended to be included within the scope of this invention.

1. A lubrication system for use with a surgical instrument, comprising:
a first fluid path comprising a first fluid inlet, and a first fluid outlet wherein the first fluid outlet does not contact a lubricant; and
a second fluid path comprising a second fluid inlet extending into the lubricant, a filtering system, and a controlled path, wherein a pressurized fluid flows through the first fluid path, causing at least a portion of the lubricant to flow through the second fluid path.

2. The lubrication system of claim 1 wherein the controlled path is a capillary tube.

3. The lubrication system of claim 1 wherein the first fluid inlet has a lower altitude than that of the controlled path.

4. The lubrication system of claim 3 wherein the first fluid outlet has a higher altitude than that of the second fluid inlet.

5. The lubrication system of claim 1 further comprising a large-diameter channel wherein the pressurized fluid flows from the large-diameter channel into the first fluid path.

6. The lubrication system of claim 1 wherein the second fluid path further comprises a small-diameter channel.

7. The lubrication system of claim 1 wherein the first fluid path further comprises a distribution route between the first fluid inlet and the first fluid outlet.

8. The lubrication system of claim 1 wherein the filtering system comprises a screen mesh.

9. The lubrication system of claim 8 wherein each opening of the screen mesh is smaller than the opening of the controlled path.

10. The lubrication system of claim 1 wherein the pressurized fluid enters into the second fluid outlet if a seal around the second fluid outlet fails.

11. The lubrication system of claim 1 wherein the pressurized fluid comprises pressurized air.

12. The lubrication system of claim 1 wherein the lubricant comprises oil.

13. A surgical system comprising a pneumatically powered surgical instrument with a motor, a pneumatic supply system for selectively providing a pressurized fluid to the motor, and a lubrication system connected to the pneumatic supply system for providing lubrication to the motor, wherein the lubrication system comprises:
a first enclosure positioned inline with a selectively pressurized fluid path of the pneumatic supply system, the first enclosure comprising a large-diameter channel and a small-diameter channel;
a second enclosure forming a chamber for containing a lubricant;
a first fluid path comprising a first fluid inlet coupled with the large-diameter channel, and a first fluid outlet extending into a first portion of the chamber; and
a second fluid path comprising a second fluid inlet extending into a second portion of the chamber, a controlled path, and a second fluid outlet coupled with the small-diameter channel,
wherein, when the pneumatic supply system provides the pressurized fluid to the motor, at least a portion of the pressurized fluid flows from the large-diameter channel, through the first fluid path, and into the chamber, thereby causing at least a portion of the lubricant to flow through the second fluid path and into the small-diameter channel, and
wherein the first and second portions of the chamber are positioned so that lubricant moves away from the first portion and towards the second portion even when the pneumatic supply system is not providing the pressurized fluid to the motor.

14. The surgical system of claim 13 wherein the controlled path is a capillary tube.

15. The surgical system of claim 13 wherein the first fluid outlet does not contact the lubricant.

16. The surgical system of claim 13 wherein the lubrication system further comprises:
a cross-over mechanism for positioning the first outlet closer to the second outlet than to the first inlet, and for positioning the second inlet closer to the first inlet than to the second outlet.

17. The surgical system of claim 13 wherein the second fluid path includes a filtering system.

18. The surgical system of claim 17 wherein the filtering system is a screen mesh and each opening of the screen mesh is smaller than an opening of the controlled path.

19. An inline oiler for use with a pneumatically powered surgical instrument, comprising:
a first inlet for receiving pressurized air;
a first outlet for providing the pressurized air into an enclosure wherein the enclosure comprises a lubricant;
a second inlet for receiving at least a portion of the lubricant wherein the second inlet extends into the lubricant; and
a capillary tube connected to the second inlet, the capillary tube having a predetermined inside diameter and a predetermined length for controlling a flow of the filtered lubricant.

20. The inline oiler of claim 19 wherein the predetermined inside diameter is about 1/5000 inch, and the predetermined length is about 1/10 inch.
